(19) United States
(12) Patent Application Publication
(10) Pub. No.: US 2006/0165701 A1
(43) Pub. Date: Jul. 27, 2006

(54) Agents for the diagnosis and treatment of tumors that expose alerted proteins on the cell surface

(75) Inventor: Christoph De Haen, Milano (IT)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: BRACCO IMAGING S.P.A., MILAN (IT)

(21) Appl. No.: 10/535,008

(22) PCT Filed: Nov. 13, 2003
(86) PCT No.: PCT/EP03/12699

§ 371 Date: Feb. 2, 2006

(30) Foreign Application Priority Data
Nov. 14, 2002 (IT) MI2002A 002411

(51) Int. Cl. A61K 39/395; A61K 31/715

(52) U.S. Cl. 424/155.1; 514/2; 514/54

(57) ABSTRACT

The present invention relates to agents for the diagnosis and treatment of tumours that expose altered proteins on the cell surface.

The present invention relates to agents for the diagnosis and treatment of tumours that expose altered proteins on the cell surface.

### BACKGROUND OF THE INVENTION

Some tumours expose on the cell surface proteins structurally altered as a result of somatic mutations. Tumours may expose structurally altered proteins also as a result of splicing variations, altered post-translational modification or partial degradation.

One of the most frequently studied families of altered proteins exposed on the surface of tumour cells derives from E-cadherin, a calcium-dependent cell adhesion molecule firmly anchored in the cytoplasmic membrane. More than 33 distinct somatically mutated forms of E-cadherin have been identified in infiltrative lobular breast cancer (Berx et. al., Hum. Mutat. 12: 226-237, 1998; Becker et al., Hum. Mutat. 13: 171, 1999). Most of these mutated forms are truncated proteins resulting from out of frame deletion mutations. Normally tumors in each patient only display one particular mutated form of E-cadherin

Human gastric tumours of the diffuse type have been described to frequently express somatically mutated E-cadherins. In this tumour, besides point mutations leading to the replacement of single amino acids, the mutations often involve an exon-skipping in-frame deletion, leading to a minimally shortened and regionally altered amino acid sequence. Such in-frame deletions have been observed in correspondence of at least 9 of the 16 exons in the E-cadherin gene. Deletions at exon 8 or 9 are by far the most frequent, followed by deletions at exon 10 and 7. These mutations are specific for tumour cells, and are never present in healthy cells; they consequently constitute an ideal target for immunotherapeutic approaches. Identification of the particular type of mutated E-cadherin present in the tumours of a patient, requires corresponding immunodiagnostic approaches.

U.S. Pat. No. 6,447,776 and EP0821060 A2 disclose monoclonal antibodies which specifically recognise mutated forms of E-cadherins. They also disclose a diagnostic or therapeutic agent in which one of these antibodies (recognition unit) is conjugated with a diagnostic radiation source (diagnostic-signal-generating unit), a therapeutic radiation source (therapeutic effect-generating unit) or a toxin (therapeutic effect generating unit). Mixtures of at least two of the disclosed agents are claimed.

Application of a product in which the therapeutic-effect-generating unit was the alpha particle-emitting radioisotope, ²¹³Bi, used for locoregional radioimmunotherapy of murine tumours expressing altered E-cadherins, was described by Senekowitsch-Schmidtke et al. in “Ninth Conference on Cancer Therapy with Antibodies and Immunoconjugates”, Abstract 21, Oct. 24-26, 2002, Princeton, N.J. Some of the same authors, in an earlier paper (Becker et al., “Molecular Targets and Cancer Therapeutics”, Miami Beach, Fla., 29 Oct.-2 Nov. 2001), proposed the use of a conjugate of a cytotoxic agent (toxin) with a monoclonal antibody able to recognise a particular E-cadherin mutant for personalised treatment of patients suffering from tumours characterised by that somatic mutation.

These approaches, which require the preparation of a distinct product for each particular mutation found in a population of patients, may be promising in some cases, but is limited by the cost problem associated with the development and production of multiple personalised drugs, i.e. a separate drug for as many types of E-cadherin mutations as one would like to be able to diagnose and/or treat therapeutically. In principle, administration of a mixture of such products targeted to all, or at least the majority of possible mutated E-cadherins, as claimed in U.S. Pat. No. 6,447,776, would partially get around the cost problem. However, this solution for the cost problem is not acceptable from a safety risk point of view, because products in the mixture not specific for the mutatated E-cadherin on the patient's tumour cells would involve a toxicological overload, i.e. exposure to radiation or exposure to cytotoxic agent, not justified by therapeutic or diagnostic benefits; these drawbacks would prevent regulatory approval of the mixture of agents.

These arguments about costs and safety risks are equally applicable to cases other than that of E-cadherin, including cases in which the various forms of structurally altered tumour surface proteins have their origin in altered splicing, post-translational modifications or altered degradation. An example of altered post-translational modification are incomplete glycosylation as a result of altered synthesis or as a result of partial degradation, provided not all altered forms occur simultaneously in each patient. Examples of alterations due to partial degradation derive from a small number of proteolytic cleavages, typically a single cleavage, inside the amino acid sequence of the extracellular domain of a membrane protein.

The above arguments are also equally applicable to targeted agents with a diagnostic-signal-generating or therapeutic-effect-generating unit of a different kind including, without thereby limiting the possibilities, radioactive halogen atoms, chelates of α, β- or γ-emitting radioisotopes, chelates of paramagnetic metal ions, chromophores for photodynamic therapy, and cytotoxic compounds.

The present invention offers a solution to the safety risk and the cost problem. The solution involves a special polyspecific targeting agent.

Polyspecific targeting agents are agents that are capable of binding to more than one structurally distinct molecular target site. Such agents are well known in the art and can be prepared by many different methods, as summarized in US2002/0025317 A1. In short, polyspecificity can be achieved by convalently or non-covalently conjugating or biochemically fusing elements that on their own show specific binding to distinct target sites. A particular form of bispecific agent is the bispecific antibody or its F(ab′)₂ fragment, a so-called diabody. In this case heavy and light chains of two antibodies with distinct specificities are combined into a hybrid structure that recognizes with each of its halfs the distinct target sites, instead of recognizing, like in a normal antibody the same target site with two separate arms.

There exist polyspecific targeting agents of the first kind that are designed to recognize with at least one of their specificities a biological target in vivo, and with at least one other of their specificities, another molecule artificially introduced into the body. Polyspecific targeting agents of the second kind are designed to recognize multiple natural targets in vivo. Here with polyspecific targeting agents those of the second kind are meant, without thereby excluding combinations of the first and second kind.

The polyspecific targeting agents of the art have one of the following properties:

Polyspecific targeting agents of the art recognizing different target sites on the same target molecule have increased avidity and specificity of the agent for its target.

Polyspecific targeting agents of the art recognizing distinct target sites on different molecules on the same cell have increased specificity and capacity of binding to cells that display simultaneously both targets or achieve additivity or synergy in action on both targets, thereby increasing the efficacy achievable with the polyspecific agent over the one achievable with a monospecific agent.

Polyspecific targeting agents of the art recognizing target sites on distinct molecules on different cell types simultaneously present in the tissue, achieve additivity or synergy between the binding and biological effects to the different cell types.

A common characteristic of all polyspecific targeting agents of the art and their applications is the interaction of the agent with all the multiple simultaneously present target sites for which they possess specificity. The advantages of polyspecificity over monospecificity in products of the art are intrinsically linked to the availability of all the multiple distinct target sites in the same patient.

The polyspecific agent of the present invention shares with the polyspecific agent of the art the basic construction as a conjugate, covalent or not, of a polyspecific recognition unit, composed of at least two recognition molecules, and a diagnostic-signal-generating or therapeutic-effect-generating unit. However, the polyspecific agent of the present invention is distinguished from the polyspecific agent of the art by the following characteristics:

Whereas the polyspecific agent of the art possesses specificities matched in number to the number of corresponding distinct types of target sites simultaneously present in a given patient, the polyspecific agent of the present invention possesses more distinct specificities than there are corresponding distinct types of target sites in any one patient.

Whereas the polyspecific agent of the art interacts in all patient with the same combination of distinct types of target sites, the polyspecific agent of the present invention does not interact in all patients with the same combination.

Whereas the polyspecific agent of the art profits in terms of overall specificity and avidity of the diagnostic or therapeutic agent in any given patient from the presence of all the specificities, the polyspecific agent of the present invention profits in any given patient only from the presence of a subset of all available specificities.

The differences between agent of the art and agent of the present invention is particularly pronounced in the special but most useful case, where each patient displays only a single abnormal protein (e.g. the case of E-cadherin) and the polyspecific agent of the invention utilizes in each patient only a single specificity from among its multiple ones. In this case multispecificity makes no contribution to increased specificity and avidity of polyspecific agent over monospecific analogue.

The present invention embodies the surprising realization that a diagnostic or therapeutic agent of the invention, i.e. a polyspecific targeting agent with N distinct specificities is advantageous
a) with respect to a mixture of N monospecific agents in terms of the risk to the patient, when it utilizes only a number smaller than N of its N specificities, especially when it utilizes only a single of its N specificities, in any given patient.
b) with respect to N separate monospecific agents in terms of drug development and production costs even when it utilizes only a single of its multiple specificities in any given patient.

A risk-related advantage of the product of the invention to the patient arises provided the following three conditions are met simultaneously:
1) The polyspecific recognition unit possesses N distinct target specificities, each specific for another of the various altered forms that a given protein in a tumour subtype can assume in a population of patients.
2) Each patient displays on its tumour, among the N altered forms of the protein recognized by the polyspecific recognition unit, only a number smaller than N of them, typically a single one.
3) The diagnostic-signal-generating unit or therapeutic-effect-generating unit has some toxic effects on or constitutes a risk to healthy tissue or the organism as a whole, radiation exposure being included in such risks.

### DESCRIPTION OF THE INVENTION

The first aspect of the invention relates to an agent for the diagnosis or treatment of tumours that in an individual patient exposes on the cell surface only a subset of the different, characteristic altered forms that a given protein of said tumour can take, said protein deriving from alterations of a normal form present in healthy tissue, said agent comprising:
a. a polyspecific recognition unit consisting of a recognition molecule specific for a first of said altered forms of the protein, conjugated with at least one other recognition molecule which recognises a different of said altered forms of the same protein not simultaneously present on the tumour,
b. at least one diagnostic-signal-generating or therapeutic-effect generating unit which supplies a diagnostic signal or therapeutic effect, conjugated with or included in said polyspecific recognition unit.

The invention also relates to diagnostic or pharmaceutical compositions containing a polyspecific agent as defined above, in admixture with a suitable vehicle.

### DETAILED DESCRIPTION OF THE INVENTION

The term “altered protein” means a protein with a structural alteration or modification, as will be specified in detail below.

Antibodies or fragments thereof able to recognise and specifically bind the altered proteins expressed by tumours can be used as recognition molecule according to the invention.

Fab, Fab′, F(ab′)₂ or scFv antibody fragments and derivatives are particularly preferred. Diabodies and their derivatives are also preferred. Alternatively, polypeptides, proteins, polysaccharides or other molecules with affinity for said altered proteins can be used.

These recognition molecules can be conjugated by chemical methods, using conventional polyfunctional reagents commonly employed in the field. The same methods can be used to chemically conjugate the recognition molecules or the entire polyspecific recognition unit with the diagnostic-signal-generating or therapeutic-effect-generating unit. Alternatively, the diagnostic-signal-generating or therapeutic-effect-generating unit may be conjugated to one of the recognition molecules by expression of genes fused by recombinant DNA techniques. For example a the gene for a proteic toxin may be fused with the gene of one of the two genes expressing the light or the heavy chain of immunoglobulin Fab fragments. Polyspecific recognition units can also be constructed fusing genes coding for multiple scFv through suitable linkers.

A special case of a polyspecific recognition unit suitable for the construction of agents of this invention is the diabody, in which the conjugation chemistry between recognition units with distinct specificities is based on the spontaneous reformation of disulfide bridges in orthologous positions during reoxydation of a mixture of two partially reduced antibodies or F(ab′)₂ fragments with different specificities. Preparation of diabodies is well known art (EP404097; WO93/11161; Hollinger et al., Proc. Natl. Acad. Sci. USA 90: 6444-6448, 1993). Diabodies or their F(ab′)₂ fragments by themselves can serve as recognition unit of the invention, or they can be used as individual recognition molecules of a larger polyspecific recognition unit.

The altered proteins expressed or exposed by tumours which can be recognised by the agents according to the invention typically present one or more mutations, point mutations, deletions, insertions or truncations, absence of post-translational modifications, altered post-translational modifications or effects of partial degradation.

Preferred examples of said altered proteins are the proteins known as E-cadherins, which in diffuse gastric tumours often present with in-frame deletions in various exons, deletions that are frequently accompanied by the creation of novel antigenic sequencies of amino acids. A preferred agent according to the invention is therefore constituted by a polyspecific agent composed of a first monoclonal antibody or a fragment or derivative thereof which recognizes E-cadherin with deletion mutation in exon 8, conjugated to second monoclonal antibody or fragment or derivative thereof which recognizes E-cadherin with a deletion mutation in exon 9, and further conjugated to a diagnostic-signal-generating or therapeutic-effect-generating unit of the kind specified below.

Said diagnostic-signal-generating or therapeutic-effect-generating unit can be covalently bound directly, or through a suitable linker, to one of the recognition molecules of the polyspecific recognition unit. Alternatively it may be covalently bound to the linker between the multiple recognition molecules or it may be integral part of the linker.

In another embodiment of the invention the diagnostic-signal-generating or therapeutic-effect-generating unit can be conjugated covalently with biotin, in which case the polspecific recognition unit will be conjugated covalently with avidin or streptavidin. Alternatively, the diagnostic-signal-generating or therapeutic-effect-generating unit can be conjugated covalently with avidin or streptavidin, in which case the polyspecific recognition unit will be conjugated covalently with biotin.

Covalent conjugation between the multiple recognition molecules and between the polyspecific recognition unit and the diagnostic-signal-generating and therapeutic-effect-generating unit is preferably obtained by reactions involving free sulfhydryl groups naturally present or generated by partial reduction of available disulfide bridges. The reagents are preferably selected from among compounds having one of the following residues: maleimino, iodoacetyl, 2,4-dinitro-fluorophenyl, pentafluorophenyl. Linkers containing multiple maleimide groups capable of reacting with free sulfhydryl groups, thereby allowing the conjugation of recognition molecules among themselves and their conjugation with the diagnostic-signal-generating or therapeutic-effect-generating unit, as well as reaction conditions for achieving conjugation, have been described for example in Smith B J et al.: Bioconjugate Chem. 12, 750-756, 2001. However, the covalent conjugation required by the present invention can also be achieved with chemistry involving other functional groups on the various components, such as OH, —NH₂ and —COOH groups, using chemistry well known in the art.

As the diagnostic-signal-generating or therapeutic-effect-generating unit can be designed to contain several of said functional groups, it can itself act as linker between the specific recognition molecules.

The diagnostic-signal-generating or therapeutic-effect-generating unit can be selected from among radioactive halogens, chelates of radioactive isotopes or paramagnetic metal ions, particles of iron oxide, stabilised microbubbles, fluorescent or phosphorescent compounds, near-infrared radiation-absorbing compounds, cytotoxic compounds, toxins, or photodynamic compounds able to generate reduced oxygen species or singlet oxygen species by irradiation, without thereby limiting the scope of the invention.

The radioactive isotope is preferably selected from among halogen isotopes ¹²³I, ¹²⁴I, ¹²⁵I, ¹³¹I, ⁷⁵Br, ⁷⁶Br, ⁷⁷Br and ⁸²Br or radioactive isotopes of other elements such as ^{99m}Tc, ¹¹¹In, ²⁰³Pb, ⁶⁶Ga, ⁶⁷Ga, ⁶⁸Ga, ¹⁶¹Tb, ⁷²As, ^{113m}In, ⁹⁷Ru, ⁶²Cu, ⁶⁴Cu, ⁶⁷Cu, ⁵²Fe, ^{52m}Mn, ⁵¹Cr, ¹⁸⁶Re, ¹⁸⁸Re, ⁷⁷As, ⁹⁰Y, ¹⁶⁹Er, ¹²¹Sn, ¹²⁷Te, ¹⁴²Pr, ¹⁴³Pr, ¹⁹⁸Au, ¹⁹⁹Au, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ⁴⁷Sc, ¹⁴⁰La, ²¹²Bi, ²¹¹At, ²¹³Bi, ²¹²Pb, ²²⁵Ac, ²²³Ra, ²²⁴Ra and ²²⁷Th. In some cases the same isotope allows diagnosis and treatment. For diagnostic applications, in particular Magnetic Resonance Imaging (MRI) techniques, a chelate of a paramagnetic metal selected from among the metal elements having an atomic number of 21-29, 39, 42, 44, 49 or 57-83 will be used. Chelates of the metal ions Gd³⁺, Fe³⁺, Eu³⁺, Dy³⁺, La³⁺, Yb³⁺ and Mn²⁺ are preferred.

Chelating groups are chosen from among the large number described in the art to be suitable for imaging or radiotherapy with the chosen metal ion and/or isotope when conjugated to a targeting agent. Obviously also polyspecific agents of the presently described kind containing novel chelating groups fall within the scope of the present invention.

Chelating groups can be conjugated to the recognition molecule either directly or by means of reactive groups such as maleimide, bis-maleimide, lysine residues and the like.

Examples of cytotoxic compounds are also residues of known antitumoral compounds, in particular residues with alkylating activity such as cyclophosphamide, chlorambucil, or natural or synthetic toxins.

For the proposed therapeutic and diagnostic uses, the agents according to the invention will be suitably formulated in the form of compositions in admixture with an appropriate vehicle.

The doses can be determined by skilled persons in the field on the basis of the pharmacokinetic and toxicological characteristics of the selected agent, as well as the type of application involved. Established guidelines which aid determination of the dose by analogy with the immunoconjugates and paramagnetic contrast agents already available for therapeutic and diagnostic applications are also available. For example, when the necessary quantity of ion, radioactive compound or paramagnetic metal has been determined, the quantity of the agent according to the invention can be determined by means of a simple stoichiometric calculation. The compositions according to the invention will preferably be in the form of solutions or suspensions in sterile vehicles suitable for parenteral administration, in particular intravenous, intraperitoneal or intramuscular administration.

The compositions according to the invention may also be supplied in the form of kits comprising:
a. the unit able to provide a diagnostic signal or therapeutic effect, covalently conjugated with biotin, and
b. the recognition unit covalently conjugated with avidin or streptavidin or, alternatively,
c. the unit able to provide a diagnostic signal or therapeutic effect, covalently conjugated with avidin or streptavidin, and
d. a recognition unit covalently conjugated with biotin.

In this case, separate administration of components a and b will allow in vivo formation of the agent according to the invention.

The following examples illustrate the invention in greater detail.

### EXAMPLE 1

Synthesis of a bis(maleimide) Derivative of DTPA (Compound 9).

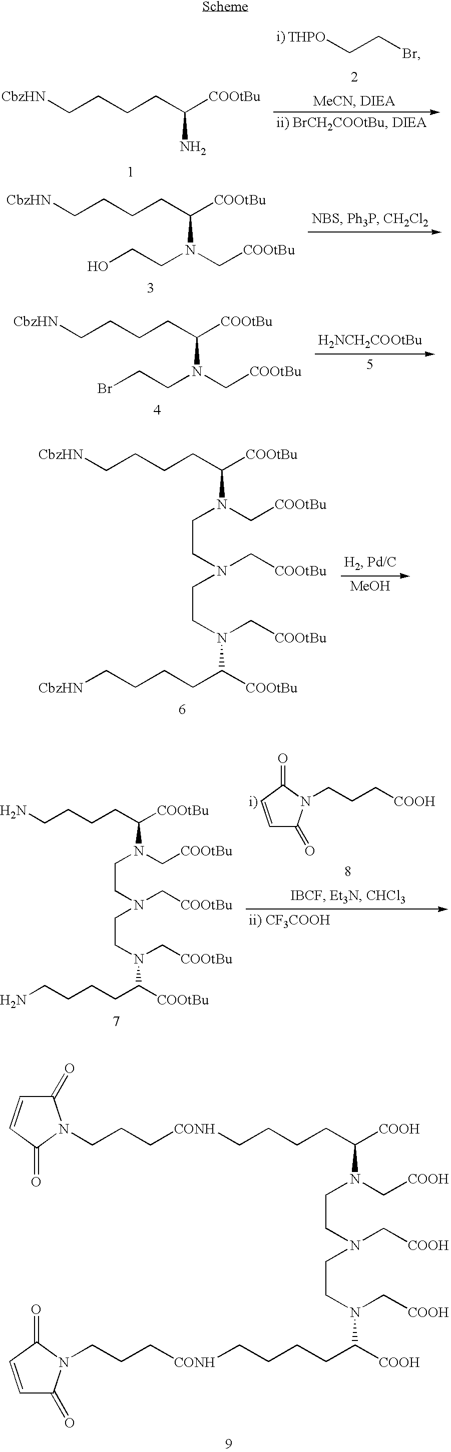

Compound 3

A solution of N⁶-[(phenylmethoxy)carbonyl]-L-lysine t-butyl ester (compound 1) (prepared according to Bioconjugate Chem. 10: 137-140, 1999) (100 mmol), 2-(2-bromoethoxy)tetrahydropyran (compound 2) (prepared according to J. Org. Chem. 51: 752-755, 1986) (135 mmol) and diisopropylethylamine (100 mmol) in MeCN is maintained under reflux for 14 h. t-butyl-bromoacetate (120 mmol) and more diisopropylethylamine (100 mmol) are added, and the mixture is maintained under reflux for a further 2 h. The solution is then evaporated to give a residue which is dissolved in Et₂O and washed with water, 1 N HCl, 1 N NaOH and water. The solution is evaporated, the residue is re-dissolved in MeOH, and 2 N HCl is added. After agitation for 2 h, 2 N NaOH is added until pH 7 is reached, then the solution is evaporated to eliminate the MeOH, and Et₂O is added to extract the product. The organic solution is separated, dried over Na₂SO₄ and evaporated to give crude compound 3, which is purified by flash chromatography.

The ¹H-NMR, ¹³C-NMR, MS and IR spectra proved consistent with the structure indicated.

Compound 4

N-Bromosuccinimide (52 mmol), in portions, is added to a solution of compound 3 (40 mmol) and triphenylphosphine (52 mmol) in CH₂Cl₂ cooled to 0° C., under stirring. The temperature of the solution is allowed to rise to room temperature, and it is washed after 4 h with water, 5% NaHCO₃ and water. The organic solution is dried (Na₂SO₄) and evaporated. The residue is purified by flash chromatography to give compound 4.

The ¹H-NMR, ¹³C-NMR, MS and IR spectra proved consistent with the structure indicated.

Compound 6

A biphasic mixture of compound 4 (22 mmol) and glycine t-butyl ester hydrochloride (compound 5) (commercial product) (10.4 mmol) in MeCN and 2 M phosphate buffer at pH 8 is stirred vigorously. After 24 h the two phases are separated and the aqueous phase is replaced by fresh 2 M phosphate buffer. After stirring for a further 24 h, the organic phase is separated and evaporated. The residue is purified by flash chromatography to give compound 6.

The ¹H-NMR, ¹³C-NMR, MS and IR spectra proved consistent with the structure indicated.

Compound 7

Pd/C (10%) is added to a solution of compound 6 in methanol, and the suspension is agitated for 6 h in a hydrogen atmosphere (1 atm; 20° C.). The resulting mixture is filtered and evaporated to give compound 7.

The ¹H-NMR, ¹³C-NMR, MS and IR spectra proved consistent with the structure indicated.

Compound 9

Isobutyl chloroformate (13 mmol) is added dropwise, under agitation, to a solution of 4-maleimidobutyric acid (compound 8) (12 mmol) and Et₃N (13 mmol) in THF at −15° C. under a hydrogen atmosphere. A solution of compound 7 (5 mmol) in THF is added dropwise after 30 min. After a further 30 min at −15° C., the temperature of the reaction mixture is allowed to rise to room temperature, and agitation is continued for 4 h. The solution is then evaporated and the residue dissolved in EtOAc and washed with water. The organic phase is dried (Na₂SO₄) and evaporated. The residue is dissolved in CH₂Cl₂, and CF₃COOH (100 mmol) is added. After 16 h the solution is evaporated, the residue is taken up with fresh CF₃COOH, and the resulting solution is kept under stirring for a further 6 h. The solution is then evaporated and the residue is purified by through elution on a resin (Amberlite® XAD 16.00T) with an MeCN/water gradient. The fractions containing the pure product are combined and evaporated to give compound 9.

The ¹H-NMR, ¹³C-NMR, MS and IR spectra proved consistent with the structure indicated.

### EXAMPLE 2

Conjugation of Two Different Fab Fragments with a Single Molecule of Compound 9 (Compound Fab1-c9-Fab2)

A volume, V, of a 2 mM solution of tris-carboxyethylphosphine (TCEP) is prepared by 1 to 250 dilution of the 0.5 M commercial product (Pierce) in a thoroughly de-aerated pH=7 buffer containing 50 mM Tris-HCl and 5 mM EDTA. This solution is then added to an equivalent volume, V, of a 10 μM solution of a first human anti-Herpes simplex recombinant Fab fragment (Fab1), prepared according to Cattani et al. (J. Clin. Microbiol. 35: 1504.1509, 1997) and incubated for 30 min at 37° C. Half a volume (V/2) of a 50 mM solution of compound 9 in 0.1 M acetate buffer at pH=5 is then added, and the reaction mixture is maintained at 37° C. for 1 h. The reaction is then complete, and the surplus reagents is removed with conventional separation technologies such as dialysis or gel filtration.

For analysis purposes, a sample is injected into a TSK-G2000SW-XL size exclusion column, and this allows the demonstration that the majority of the protein remains approximately the size of a Fab fragment. Only a small part is approximately the size of two Fab fragments. The product which has the same size as one Fab is purified on a Sephacryl S-200HR size-exclusion column (Amersham Biosciences). The recovered material is further purified on a cation exchange column (Resource-S, Amersham Biosciences) and eluted with a saline gradient. The peak corresponding to the 1:1 conjugate of Fab1 with compound 9 (Fab1-c9) is collected and set aside.

A volume, V, of a 2 mM solution of TCEP is prepared by 1 to 250 dilution of the 0.5 M commercial product (Pierce) in a thoroughly de-aerated pH=7 buffer containing 50 mM Tris-HCl and 5 mM EDTA. This solution is added to an equivalent volume, V, of a 10 μM solution of a second Fab fragment (Fab2), specific for tetanus toxin, isolated by digestion with papain of a commercial antibody (Terbutalin, Baxter AG, Vienna), and incubated for 30 min at 37° C., yielding the reduced Fab2.

A molar quantity of Fab1-c9 equivalent to that of the reduced Fab2 is then added as 10 μM solution in 0.1 M acetate buffer at pH=5, and the reaction mixture is maintained at 37° C. for 1 h.

The reaction mixture is separated on a Sephacryl S-200HR size-exclusion column, and material of a size approximately equivalent to two Fab fragments is isolated. The final material, called Fab1-c9-Fab2, is proven to be homogeneous when tested on a TSK G2000SW-XL analytical size-exclusion column.

### EXAMPLE 3

Conjugate with Two Different Anti-Mutated E-Cadherin Fab Fragments (Compound Fab3-c9-Fab4)

Fab fragments of rat antibody fully specific for E-cadherins with mutation in both exon 8 (Fab3) and exon 9 (Fab4) are prepared according to the method of Becker et al. (Poster #648, Molecular Targets and Cancer Therapeutics. Miami Beach, Fla., Oct. 29-Nov. 2, 2001). These Fabs do not interact with natural E-cadherins. The Fab3-c9-Fab4 conjugate is prepared according to the teaching of example 2.

### EXAMPLE 4

Labelling of Fab1-c9-Fab2 with ¹¹¹In

The conjugate described in Example 2, Fab1-c9-Fab2, is formulated at the concentration of 0.25 mg/mL in pH 6 acetate buffer. The Indium-111 chloride is available from Amersham at the concentration of 0.2 μg/mL (10 mCi/mL). Labelling is performed by incubation at room temperature for 30 min. Labelling efficiency is tested by thin-layer chromatography with ITLC-SG strips (Gelman Laboratories), using an 0.9% solution of NaCl as mobile phase.

The reaction mixture is also analysed through HPLC by size-exclusion chromatography with a TSK-gel G3000 column; phosphate-buffered saline (PBS) added with 0.2 M NaCl was used as eluent. The eluate was monitored by UV detector at the wavelengths of 280 and 254 nm and a radiometric detector placed in series with the UV detector. The radiopharmaceutical, ¹¹¹In-Fab1-c9-Fab2, gives a single radioactivity peak corresponding to the unlabelled protein. 98% labelling efficiency is obtained with a Fab1-c9-Fab2/¹¹¹InCl₃ stoichiometric molar ratio of 3/1.

### EXAMPLE 5

Labelling of Fab3-c9-Fab4 with ¹⁷⁷Lu

Using the conjugate Fab3-c9-Fab4 and lutetium-177 chloride in molar proportions 1:0.9, the procedure described in example 4 supplies a conjugate labelled with lutetium-177. The product can be used in radioimmunotherapy of metastases deriving from stomach tumours that bear E-cadherin with a mutation deletion in either exon 8 or exon 9, but never bear both mutated E-cadherins simultaneously or both mutations in the same E-cadherin. The same product may be used for both cases without any disadvantage in terms of radiation dose compared with a product with a single specificity for one or the other of the mutated E-cadherins, and with a net advantage in terms of radiation dose when compared with a mixture of the individual Fab fragments each labelled with Lu-177.

### EXAMPLE 6

Scintigraphy of Herpes Simplex Infection of the Eye of a Rabbit with the Product Described in Example 2, Labelled with ¹¹¹In-Fab1-c9-Fab2

Corneal de-epithelialisation of one of the eyeballs was performed on adult albino rabbits weighing 3 kg, after topical anaesthesia with naropin. The virus was then inoculated by instillation into the conjunctival sac of the damaged eye during 180 min of 100 to 150 μL of a solution containing 1×10⁶ plaque-forming units of clinically isolated Herpes Simplex Virus type 1 (HSV-1). Keratitis in the form of a dendritic ulcer was clinically manifest in all the animals after 36 to 48 h. The animals were clinically monitored thereafter, with daily ophthalmological examinations for 2 weeks. No complications were observed in any of the animals.

A portable gamma chamber with high spatial resolution was used for the scintigraphic evaluation. Compound ¹¹¹In-Fab 1-c9-Fab2 prepared according to Example 4 at the dose of 8 μg/kg of body weight was administered 48 h after the infection; scintigraphic evaluation was performed 3, 6, 24 and 48 h after administration. The animals were then sacrificed, and both eyeballs were removed.

In all three rabbits studied, the radioactivity of the diseased eye proved to be about 8 times stronger than that of the healthy eye; the greatest difference in enhancement was demonstrated by the measurements taken after 3 and 6 h, whereas the contrastographic differences proved lower in the measurements taken after 24 and 48 h.

This in vivo test demonstrated that ¹¹¹In-Fab1-c9-Fab has suitable characteristics to visualise herpes infections. It also demonstrated that the presence of the second recognition molecule, tetanus anti-toxin, in the same conjugate, does not prevent anti-herpetic functionality.

### EXAMPLE 7

Assay of Tetanus Anti-Toxin Activity for the Product Fab1-c9-Fab2.

Tetanus anti-toxin activity was determined with a commercial ELISA kit (Tetanus ELISA IgG kit, ICN Diagnostic) in 96-well plates, the secondary antibody being replaced with a Fab human antibody conjugated with horseradish peroxidase (Pierce), and visualised with TMB colorimetric substrate (Sigma). The activity of the product Fab1-c9-Fab2 proved equal to that of Fab isolated from the preparation of starting antibodies (Tetabulin, Baxter), analysed at equivalent molarities (molecular weight: about 49,000 for the isolated Fab and about 100,000 for Fab1-c9-Fab2).

This in vitro test demonstrates that the functionality of both recognition molecules is maintained after conjugation, without any substantial interference between them.

### EXAMPLE 8

Synthesis of a Biotin-Substituted bis-maleimide Compound

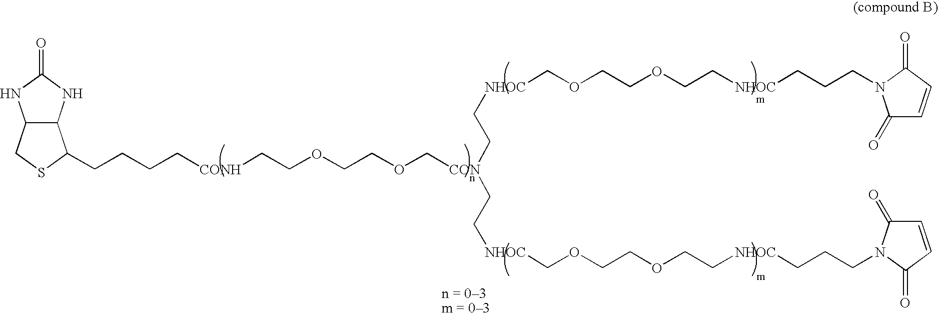

The compound having the formula shown above, with m=n=1 (compound B), was prepared from 1,7-bis(trifluoroacetyl)-1,4,7-triazaheptane (prepared according to U.S. Pat. No. 5,514,810) by coupling with N-t-butoxycarbonyl-8-amino-3,6-dioxaoctanoic acid (Org. Prep. Proced. Int. 2002, 34, 326-331) in the presence of N,N,N′,N′-tetramethyl-O-(1H-benzotriazol-1-yl)uronium hexafluorophosphate (HBTU) in DMF. The product obtained was deprotected with K₂CO₃ in MeOH/H₂O, and the diamine obtained was condensed to N-fluorenylmethoxycarbonyl-8-amino-3,6-dioxaoctanoic acid using HBTU in DMF. This product was deprotected with piperidine to give the corresponding diamine, which was reacted with 2 molar equivalents of 4-maleimidobutyric acid N-hydroxysuccinimidyl ester. The product obtained was deprotected with CF₃COOH and then reacted with biotin N-hydroxysuccinimidyl ester to give the end product, B.

### EXAMPLE 9

Conjugate with Two Different Anti-Mutated E-Cadherin Fab Fragments Bearing a Biotin Residue (Fab1-B-Fab2)

With preparation methods analogous to those described in Example 2, but using compound B instead of compound 9, a product with a biotinyl residue, called Fab1-B-Fab2, is obtained. This compound can be used for the detection and treatment of lesions according to U.S. Pat. No. 5,482,698.

### EXAMPLE 10

Preparation of a Recombinant Fusion Protein Between Fab and a Fragment of Pseudomonas Exotoxin, Toxin-Fab1.

The plasmid used to produce the anti-Herpes simplex human Fab described in example 2 contains cistrons for the heavy chain and the light chain under the control of two identical promoters, from 5′ and 3′ respectively. Following the method described in U.S. Pat. No. 6,099,842 and using normal genetic engineering techniques, a codifying sequence for a fragment of Pseudomonas exotoxin with a molecular weight of 40,000, called PE40, is inserted into the described plasmid contiguously with the end of the gene codifying the light chain. The modified plasmid serves to produce a recombinant fusion protein between the original Fab, Fab1, and the toxin fragment PE40 in E. coli; this construct is called Toxin-Fab 1.

### EXAMPLE 11

Preparation of a Conjugate Between a Fusion Protein Incorporating a Fab and an Exotoxin Fragment (Toxin-Fab1) and a Fab of Other Specificity, Toxin-Fab1-c9-Fab2.

A conjugate between Toxin-Fab1 and a normal Fab, Fab2, with different specificity from Toxin-Fab, is prepared according to example 2 to obtain a product called Toxin-Fab1-c9-Fab2. As the fusion of PE40 in the carboxy-terminal position of the light chain can leave the affinity of the binging site of an antibody for the target site intact (U.S. Pat. No. 6,099,842), Toxin-Fab1-c9-Fab2 will continue to recognise cells infected by Herpes simplex and cause their death.

### EXAMPLE 12

Preparation of a Conjugate Between a First Fab Specific for a Mutation of E-Cadherin and Fused with a Toxin (Toxin-Fab1), and a Second Fab Specific for a Second Mutation of E-Cadherin.

By following example 10 and using the system employed by Becker et al. (Poster #648, Molecular Targets and Cancer Therapeutics. Miami Beach, Fla., Oct. 29-Nov. 2, 2001) to produce the two different anti-mutant E-cadherin Fab in E. coli, a recombinant fusion protein is obtained between Fab specific for the E-cadherin mutated in exon 8 (Fab3) and a fragment of Pseudomonas exotoxin, PE40, called Toxin-Fab3. By following the procedures described in example 11, but using Toxin-Fab3 and the Fab anti-E-cadherin mutated in exon 9 (Fab4), a conjugate called Toxin-Fab3-c9-Fab4 is obtained. This product promises to be useful to treat patients with stomach carcinoma characterised by deletion mutations in either exon 8 or in exon 9 of E-cadherin, these mutated E-cadherins not occurring simultaneously in individual patients. In a patient bearing a tumor with a deletion in exon 8 of E-cadherin, the presence of a recognition molecule for E-cadherin with a deletion in exon 9 in the targeted therapeutic product Toxin-Fab3-c9-Fab4 will produce no toxic extra burden without therapeutic benefit. The single bispecific product Toxin-Fab3-c9-Fab4 will be useful for a larger population of cancer patients than a monospecific product. This reduces development and production costs relative to two separate products.

### EXAMPLE 13

Radiodiagnosis and Radiotherapy with the Products Described in Examples 3 and 5

The primary tumour was removed from a patient with a gastric tumour of the sporadic diffuse type. Immunohistological tests demonstrated that the tumour exposes an E-cadherin with deletion in exon 9. After administration of the product described in Example 3 labelled with ¹¹¹In, as in Example 4, scintigraphy reveals the location of the metastasis and the residual primary tumour. The dosimetry required for radioimmunotherapy is obtained at the same time. The assay and the image acquisition time are optimised for the patient's weight. Radioimmunological treatment is performed with the product described in Example 5, in administration regimens optimised in the clinical trials required for registration of the product.

1. An agent for the diagnosis or treatment of those tumours that in an individual patient expose on the cell surface only a number n smaller than N of N different altered forms that a given protein or glycoprotein of said tumour type can assume in a population of patients, said altered forms of the protein deriving from alterations of a normal form present in healthy tissue, said agent comprising:
a. a recognition unit consisting of a conjugate of m recognition molecules, where m is at least 2 and equal or smaller than n, and each recognition molecule is specific for a different altered form of the protein, and,
b. at least one unit which supplies a diagnostic signal or therapeutic effect, conjugated with or included in said specific recognition unit.

2. An agent as claimed in claim 1, wherein the recognition molecules are selected from among immunoglobulins or fragments thereof, polypeptides and polysaccharides.

3. An agent as claimed in claim 2, wherein at least one recognition molecules is an Fab, F(ab′) or scFv fragments.

4. An agent as claimed in claim 2, wherein the recognition molecules are conjugated to one another by means of a direct covalent bond or by means of a multipurpose linker able to form covalent bonds with the molecules, and/or as a result of the expression of fused genes with suitable linker regions.

5. An agent as claimed in claim 1, wherein at least one of the specific recognition molecules recognizes a protein altered as a result of one or more mutations.

6. An agent as claimed in claim 1, wherein at least one of the specific recognition molecules recognises a protein altered as a result of post-translational modifications, deficient post-translational modifications, absence of post-translational modifications or partial degradation.

7. An agent as claimed in claim 1, wherein one of the specific recognition molecules recognizes an E-cadherin with a deletion in exon 8 and another molecule recognises E-cadherin with a deletion in exon 9.

8. An agent as claimed in claim 1, wherein the unit able to provide a diagnostic signal or therapeutic effect is linked directly, via an avidin/biotin or streptavidin/biotin system or via a suitable covalent linker to one of the recognition molecules of the recognition unit, or to the linker that holds the recognition molecules together.

9. An agent as claimed in claim 8, wherein the unit able to provide a diagnostic signal or therapeutic effect is conjugated covalently with biotin, and the recognition unit is conjugated covalently with avidin or streptavidin.

10. An agent as claimed in claim 8, wherein the unit able to provide a diagnostic signal or therapeutic effect is conjugated covalently with avidin or streptavidin, and the recognition unit is conjugated covalently with biotin.

11. An agent as claimed in claim 1, wherein the unit able to provide a diagnostic signal or therapeutic effect is part of the bond between the recognition molecules of the recognition unit.

12. An agent as claimed in claim 1, wherein the unit able to provide a diagnostic signal or therapeutic effect is a radioactive halogen, a chelate of an radioactive isotope, a chelate of a paramagnetic metal ion, a stabilized particle of iron oxide, a stabilized microbubble, a fluorescent, phosphorescent or near-infrared radiation-absorbing compound, a cytotoxic compound, a natural or synthetic toxin, or a photodynamic compound able to generate reduced oxygen species or singlet oxygen by irradiation.

13. An agent as claimed in claim 12, wherein the radioactive halogen is selected from ¹²³I, ¹²⁴I, ¹²⁵I, ¹³¹I, ⁷⁵Br, ⁷⁶Br, ⁷⁷Br and ⁸²Br.

14. An agent as claimed in claim 12, wherein the radioactive isotope is selected from among ^{99m}Tc, ¹¹¹In, ²⁰³Pb, ⁶⁶Ga, ⁶⁷Ga, ⁶⁸Ga, ¹⁶¹Tb, ⁷²As, ^{113m}In, ⁹⁷Ru, ⁶²Cu, ⁶⁴Cu, ⁶⁷Cu, ⁵²Fe, ^{52m}Mn, ⁵¹Cr, ¹⁸⁶Re, ¹⁸⁸Re, ⁷⁷As, ⁹⁰Y, ¹⁶⁹Er, ¹²¹Sn, ¹²⁷Te, ¹⁴³Pr, ¹⁹⁸Au, ¹⁹⁹Au, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ⁴⁷Sc, ¹⁴⁰La, ²¹¹At, ²¹²Bi, ²¹³Bi, ²¹²Pb, ²²⁵ Ac, ²²³Ra, ²²⁴Ra and ²²⁷Th.

15. An agent as claimed in claim 12, wherein the paramagnetic metal is selected from the metal elements having an atomic number of 21-29, 39, 42, 44, 49 or 57-83.

16. An agent as claimed in claim 15, wherein the metal is selected from among Gd³⁺, Fe³⁺, Eu³⁺, Dy³⁺, La³⁺, Yb³⁺ and Mn²⁺.

17. An agent as claimed in claim 15, wherein the metal or isotope is chelated by chelating groups deriving from diethylenetriamine or from polyamine macrocycles, both substituted by residues bearing carboxy, phosphonic or sulphonic groups.

18. An agent as claimed in claim 1, wherein the various recognition molecules are conjugated to one another, or said recognition molecules are conjugated with the therapeutic or diagnostic unit, by reaction between sulfhydryl-reactive groups and the sulfhydryl groups present, or generated by reduction of disulfide bridges, on said units/molecules.

19. Pharmaceutical or diagnostic compositions containing an agent as claimed in claim 1, in admixture with a suitable vehicle.

20. Compositions as claimed in claim 19, in the form of a kit containing:
a. the unit able to provide a diagnostic signal or therapeutic effect, covalently conjugated with biotin, and
b. a recognition unit covalently conjugated with avidin or streptavidin.

21. Compositions as claimed in claim 19, in the form of a kit containing:
a. the unit able to provide a diagnostic signal or therapeutic effect covalently conjugated with avidin or streptavidin, and
b. a recognition unit covalently conjugated with biotin.
